(19) United States
(12) Patent Application Publication
(10) Pub. No.: US 2021/0316096 A1
(43) Pub. Date: Oct. 14, 2021

(54) PROGRAMMABLE LOGIC CONTROLLER-BASED SCALABLE VENTILATOR

(71) Applicant: RED BALLOON SECURITY, INC., New York, NY (US)

(72) Inventor: Ang Cui, New York, NY (US)

(21) Appl. No.: 17/230,743

(22) Filed: Apr. 14, 2021

Related U.S. Application Data
(60) Provisional application No. 63/009,585, filed on Apr. 14, 2020.

(51) Int. Cl. A61M 16/00; A61M 16/20; A61M 16/06; A61M 16/16; A61M 16/12

(52) U.S. Cl. CPC A61M 16/024; A61M 16/201; A61M 16/06; A61M 2205/505; A61M 16/12; A61M 16/0003; A61M 16/16

(57) ABSTRACT

In general, one aspect disclosed features a scalable ventilator system, comprising: a plurality of ventilator modules; a controller to control operation of the plurality of ventilator modules; and a station module comprising a plurality of receptacles, wherein each receptacle is configured to accept one of the plurality of ventilator modules; wherein each ventilator module comprises a plurality of solenoid valves and an input hose and an output hose, wherein each ventilator is controlled individually to provide individualized regimens based on needs of a patient corresponding to a respective ventilator.

### REFERENCE TO RELATED APPLICATION

The present application claims priority to U.S. Provisional Patent Application No. 63/009,585, filed Apr. 14, 2020 and titled “PROGRAMMABLE LOGIC CONTROLLER-BASED SCALABLE VENTILATOR,” which is incorporated herein by reference in its entirety.

### DESCRIPTION OF RELATED ART

Medical ventilators are vital components to ensure that a patient's respiratory function is continued during care and are utilized throughout hospitals, including intensive care units (ICUs) and during medical operations. Most commercial ventilators are mobile units that can be moved into different rooms throughout the hospital to assist patients in breathing. These medical ventilators are designed for a single patient, very expensive—costing tens of thousands of dollars in most cases and the time to manufacture and ship the devices can impact ventilator availability during extreme periods, such as epidemics or pandemics.

### SUMMARY

in general, one aspect disclosed features a scalable ventilator system, comprising: multiple ventilator stations each comprising: a positive air valve configured to receive air from a positive air supply, a flow regulator configured to control a rate of flow of the air, and a manifold configured to deliver the air to a patient; and a controller configured to control the positive air valves and the flow regulators.

Embodiments of the scalable ventilator system may include one or more of the following features. In some embodiments, at least one of the ventilator stations further comprises: one or more sensors configured to monitor the air, wherein the one or more sensors are communicatively coupled to the controller. In some embodiments, at least one of the ventilator stations further comprises: a humidifier configured to control a humidity of the air, wherein the humidifier is controlled by the controller. In some embodiments, at least one of the ventilator stations further comprises: a mixer configured to add one or more fluids to the air, wherein the mixer is controlled by the controller. In some embodiments, at least one of the ventilator stations further comprises: a negative air valve configured to provide the air to a negative air supply, wherein the negative air valve is controlled by the controller, Some embodiments comprise a master control panel configured to control the controller according to user inputs. In some embodiments, at least one of the ventilator stations further comprises: a local control panel configured to control a controller configured to control the positive air valve and the flow regulator of the at least one of the ventilator stations according to user inputs. In some embodiments, the controller comprises: multiple slots each connected to one of the multiple stations. In some embodiments, the controller further comprises: multiple additional slots each configured to connect to a respective additional station.

In general, one aspect disclosed features a scalable ventilator system, comprising: multiple ventilator stations each comprising: a positive air valve, a flow regulator, and a manifold configured to deliver air to a patient; a hardware processor; and a non-transitory machine-readable storage medium encoded with instructions executable by the hardware processor to perform operations comprising: operating the positive air valve to receive the air from a positive air supply, and operating the flow regulator to control a rate of flow of the air to the manifold.

Embodiments of the scalable ventilator system may include one or more of the following features. In some embodiments, the operations further comprise: receiving sensor data concerning the air from one or more sensors in at least one of the ventilator stations. In some embodiments, the operations further comprise: operating a humidifier to control a humidity of the air in at least one of the ventilator stations. In some embodiments, wherein the operations further comprise: operating a mixer to add one or more fluids to the air in at least one of the ventilator stations. In some embodiments, the operations further comprise: operating a negative air valve to provide the air to a negative air supply in at least one of the ventilator stations. In some embodiments, the operations further comprise: operating the positive air valves and the flow regulators in the ventilator stations according to user inputs at a master control panel. In some embodiments, the operations further comprise: operating the positive air valve and the flow regulator in one of the ventilator stations according to user inputs at a local control panel.

In general, one aspect disclosed features a computer-implemented method, comprising: operating positive air valves in multiple ventilator stations to provide air from a positive air supply to each of the multiple ventilator stations; receiving sensor data concerning the air from sensors in the multiple ventilator stations; and operating flow regulators in the in the multiple ventilator stations to control rates of flow of the air to respective manifolds in the multiple ventilator stations in accordance with the sensor data and respective patient treatment plans.

Embodiments of the method may include one or more of the following features. Some embodiments comprise at least one of: operating a humidifier to control a humidity of the air in at least one of the ventilator stations; and operating a mixer to add one or more fluids to the air in at least one of the ventilator stations. Some embodiments comprise operating the positive air valves and the flow regulators in the ventilator stations according to user inputs at a master control panel. Some embodiments comprise operating the positive air valve and the flow regulator in one of the ventilator stations according to user inputs at a local control panel.

In general, one aspect disclosed features a scalable ventilator system, comprising: a plurality of ventilator modules; a controller to control operation of the plurality of ventilator modules; and a station module comprising a plurality of receptacles, wherein each receptacle is configured to accept one of the plurality of ventilator modules; wherein each ventilator module comprises a plurality of solenoid valves and an input hose and an output hose, wherein each ventilator is controlled individually to provide individualized regimens based on needs of a patient corresponding to a respective ventilator.

Embodiments of the scalable ventilator system may include one or more of the following features. In some embodiments, a quantity of ventilator modules installed into their respective receptacles in the station module may be scaled to meet patient requirements.

### BRIEF DESCRIPTION OF THE DRAWINGS

The present disclosure, in accordance with one or more various embodiments, is described in detail with reference to the following figures. The figures are provided for purposes of illustration only and merely depict typical or example embodiments.

FIG. 1 is an example scalable ventilator system in accordance with various embodiments of the technology disclosed herein.

FIG. 2 is an example controller flow diagram in accordance with various embodiments of the technology disclosed herein.

FIG. 3 is an example scalable ventilator system connected to a plurality of stations in accordance with various embodiments of the technology disclosed herein.

FIG. 4 is an example computing component in accordance with various embodiments of the technology disclosed herein.

FIG. 5 is an example computing component that may be used to implement various features of embodiments described in the present disclosure.

The figures are not exhaustive and do not limit the present disclosure to the precise form disclosed.

### DETAILED DESCRIPTION

The expense and lead time for commercial medical ventilators make it difficult for hospitals and other health care facilities to maintain a sufficient stockpile to deal with massive emergency situations, such as the novel COVID-19 pandemic that is currently sweeping the world and pushing medical resources to the brink. Most commercial medical ventilators are designed and capable of helping a single patient breath when unable to breathe on their own. Some newer designs are dual-capacity, enabling two patients to be assisted by one machine, although there is generally no ability to customize operation for each patient individually.

However, the current systems cannot efficiently scale in the time necessary to properly respond in times of need. Hospitals generally cannot maintain a large enough stockpile of traditional ventilators or dual-capacity ventilators due to the high cost and the long manufacturing lead time. When the number of people requiring ventilators increases exponentially, the stockpile on hand is quickly diminished. Even using dual-capacity ventilators, in emergency situations the number of patients can quickly use up all of the available ventilators. This puts immense strain on the health care system, leaving those who need ventilators potentially failing to receive the necessary care and risking death, as well as placing additional stress on the medical professionals providing care who may need to make terrible decisions as to who should be given ventilation over others. Essentially, the situation places doctors and nurses in the unenviable position of having to decide who lives when only one ventilator is available. Moreover, commercially available medical ventilators are not easy to manufacture, and as a result, the supply cannot keep pace with a wide-ranging pandemic or emergency. Commercial ventilators comprise stand-alone units, requiring that each unit has all of the necessary components required for operation, such as embedded controllers and logical components, as well as its own self-contained vacuum pump and air compressor. Accordingly, the current market for medical ventilators is not equipped for fast, large-scale manufacturing where a disease or disaster causes unprecedented needs.

Embodiments of the technology disclosed herein are directed to a scalable and efficient ventilator system that can be used to control upwards of 64 patients. A programmable logic controller (PLC) having a central processing unit (CPU) is configured to control operation of a plurality of input modules and a plurality of output modules. Each “station” comprises two solenoid valves and two hoses, one hose as the air input and the other hose as the air output. In various embodiments, each station can be controlled by a dedicated control device, and in some embodiments a master control device can be used for overall system configuration and control. In various embodiments, one or more sensors can be added to each station to provide additional feedback to the PLC regarding a variety of parameters, including but not limited to pressure or temperature sensors. The number of slots of the PLC enable the ventilator system to scale by adding additional station components (an additional input module and output module). Through individual configuration of each station, embodiments are capable of providing individualized regimens based on each patient's individual needs.

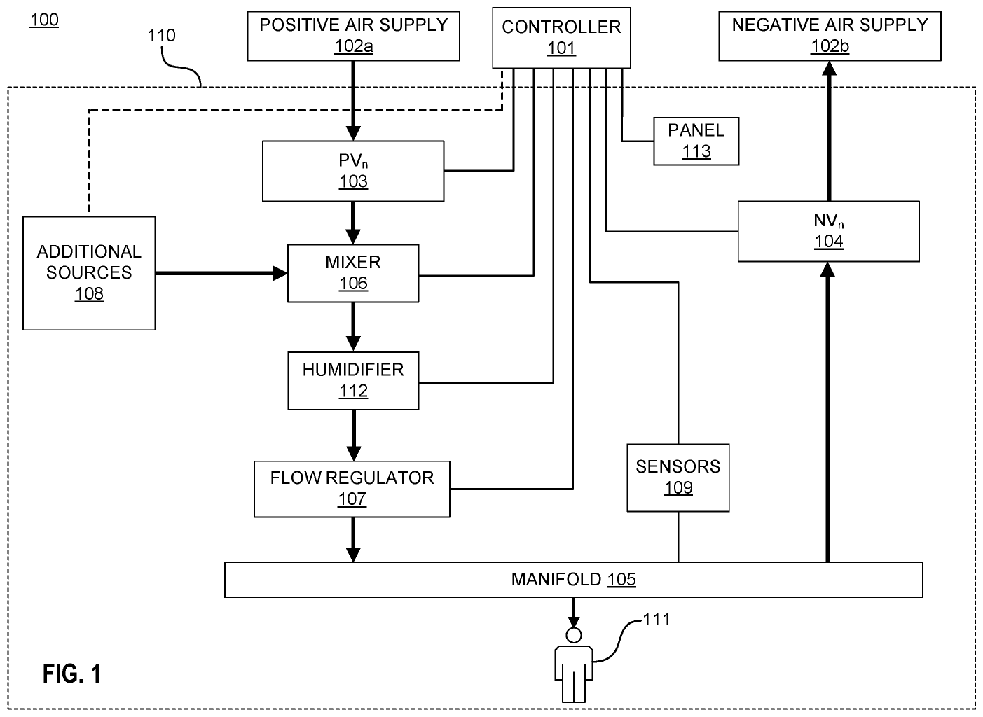

FIG. 1 illustrates an example scalable ventilator system 100 in accordance with embodiments of the technology disclosed herein. The scalable ventilator system 100 is provided for illustrative purposes only and should not be interpreted as limiting the scope of the technology disclosed herein. For purposes of FIG. 1, the arrows refer to air tubes whereas the smaller lines represent communication connections. Unless otherwise stated, the nature of the arrows and/or lines refer only to the illustrated embodiment of FIG. 1. As shown in FIG. 1, the scalable ventilator system 100 comprises a controller 101 capable of controlling a plurality of inputs and a plurality of outputs. In various embodiments, the controller 101 can comprise a processing system including one or more processors configured to control the operation of one or more components communicatively coupled to the controller 101. In some embodiments, the controller 101 can comprise one or more hardware processors, software processors, or a combination of both. In various embodiments, the controller 101 can comprise a central processing unit (CPU) of a programmable logic controller (PLC). PLCs are computers that have been ruggedized and adapted to control industrial processes requiring high reliability. The controller 101 can be configured to receive a plurality of data from a plurality of different components and process the information in real time to ensure the various components are functioning properly to achieve an associated goal.

The controller 101 can be configured to operate a plurality of different stations 110, each station 110 being associated with a set of components of the scalable ventilator system 100. Each station 110 will include all or fewer of the elements of the scalable ventilator system 100 discussed with respect to FIG. 1. Each of the components discussed herein can be connected to the controller 101 through a controller interface (not shown in FIG. 1), such as a bus interface, a communication backplane, or some other line-speed computer interface known in the art. As illustrated, each station 110 comprises at least one positive air valve (PVₙ) 103 and at least one negative air valve (NVₙ) 104 (collectively, “the air valves 103, 104”), each comprising an input and an output. Each of the at least one positive air valve 103 and the at least one negative air valve 104 are controllable components capable of controlling the amount of air being let into or out of the station 110. In various embodiments, the at least one positive air valve 103 and the at least one negative air valve 104 can comprise one or more types of controllable valve components known in the art that provide flow control through either pneumatic, electric, and/or hydraulic actuation. As a non-limiting example, the at least one positive air valve 103 and the at least one negative air valve 104 can comprise a solenoid-based valve that is communicatively coupled to and controlled by the controller 101. In various embodiments, the air valves 103, 104 can comprise one or more types of control valve assemblies capable of controlling the amount of air that passes through an output, through any of the actuation types identified above. The input of the at least one positive air valve 103 and the at least one negative air valve 104 are each coupled to a positive air supply 102a and a negative air supply 102b, respectively. In some embodiments, each of the air valves 103, 104 may be coupled to a distinct positive air supply 102a and negative air supply 102b, respectively, that is associated with the specific station 110. In other embodiments, a plurality of air valves 103, 104, each pair associated with a specific station 110, can be coupled to the same positive air supply 102a and negative air supply 102b. In some embodiments, the air valves 103, 104 may be connected to the positive air supply 102a and negative air supply 102b through an output of a positive air supply manifold (not shown in FIG. 1) and a negative air supply manifold (not shown in FIG. 1), respectively, with each air supply manifold comprising a plurality of outputs and at least one input, the at least one input connected to the positive air supply 102a and the negative air supply 102b, respectively.

The term “negative” used throughout this disclosure does not mean a value less than zero. Rather, the reference to “negative air supply” refers to an air supply having an air pressure that is lower than the air pressure of the “positive air supply.” The air pressure of the negative air supply can be higher than zero air pressure, but less than the positive air supply. During operation, the positive air supply will be greater, and the passive pressure of the patient's lungs will overcome the “negative” pressure, to push the exhalation out of the lungs and into the negative air valve input (essentially, into the negative air supply).

Focusing on the input side, each station 110 comprises a mixer 106 having a first input connected to an output of the positive air valve 103, and being communicatively coupled to the controller 101. The mixer 106 is configured to take the air from the output of the positive air valve 103 and generate the right air mixture according to the requirements of a patient 111. In various embodiments, the mixer 106 can comprise one or more additional inputs configured to enable one or more additional fluids to be mixed with the air from the positive input valve 103. One or more additional sources 108 can be connected to the one or more additional inputs of the mixer 106. In some embodiments, the additional sources 108 can comprise one or more of an oxygen supply, a nebulizer, or other source of fluid to be mixed with the air from the positive air valve 103 for the patient's treatment. In some embodiments, the additional sources 108 may be communicatively coupled to the controller 101, or can be controlled either manually or by some other component. The flow of the air mixture from an output of the mixer 106 can be passed through a humidifier 112 in various embodiments. In some situations, the mixture of input supply air from the positive air valve 103 and a fluid from one or more additional sources 108 may be overly dry, which can result in adverse effects for the patient (e.g., nose bleeds). In some embodiments, the controller 101 can configure a humidifier 112 to cause the air mixture from the mixer 106 to have a certain humidity level to overcome undesirable dryness.

In various embodiments, the flow rate of the air mixture to the patient (through the manifold 105) can be controlled by a flow regulator 107. The flow regulator 107 is configured to manage the flow rate of the air mixture. In some embodiments, the input of the flow mixture may be connected to the output of the mixer 106, while in other embodiments the input can be connected to the output of the humidifier 112. In some embodiments, the flow regulator 107 can comprise a controllable flow regulator 107, communicatively coupled to the controller 101. Where necessary, the controller 101 can increase or decrease the flow rate using the flow regulator 107 according to the patient's treatment plan. In various embodiments, the manifold 105 can provide a location where the air tube connected to an output end of the flow regulator 107 is combined with one or more additional components. In various embodiments, the manifold 105 can comprise a housing having a plurality of inputs, one of which is configured to accept the air tube connected to the output of the flow regulator 107. In various embodiments, the manifold 105 may combine the air tube from the flow regulator 107 with one or more sensors 109 and an air tube associated with an output side of the station 110 (i.e., the air tube connecting the manifold 105 to the negative air valve 104. The output of the manifold 105 can be connected to one or more types of patient air tubes commonly used with medical ventilators and known in the art. In some embodiments, the patient air tube can include a face mask configured to cover the patient's nose and mouth, whereas in other embodiments the patient air tube can comprise a intubation assembly configured to be inserted into the throat of the patient to push air in and pull air out of the patient's lungs.

In various embodiments, a plurality of sensors 109 can be communicatively coupled to the controller 101. Each of the plurality of sensors 109 can be included within or communicatively coupled to the patient's air tube to monitor the performance of the station 110 at the patient. In various embodiments, the plurality of sensors 109 can comprise one or more sensors configured to monitor data associated with air pressure, temperature, oxygen level, humidity, drug concentration, or other data point applicable to the patient's treatment plan. In various embodiments, similar sensors, like the sensors 109, can be configured to monitor the same or similar parameters of the positive air supply 102a and the negative air supply 102b, and feed this information to the controller 101. The controller 101 can utilize this information in determining how to modify the configuration of components of each station 110 according to the patient's needs. In some embodiments, one or more of the sensors 109 can include one or more logic circuits configured to determine if a change in the measured data occurs.

During operation, the controller 101 can configure the positive air valve 103 to allow a certain amount of air from the positive air supply 102a to flow into the station 110, configure the air mixture level of the mixer 106, and configure the air flow rate with the flow regulator 107. When air is input into the system through the positive air valve 103, the mixer 106 can be configured to mix the air from the positive air supply 102a with one or more additional sources, such as oxygen from an oxygen tank. The air mixture is then pushed into the patient's lungs through the patient air tube according to the treatment requirements of the patient during the first part of a cycle. After air is pushed into the patient's lungs, the air is then pulled from the patient's lungs by the negative air valve 104 during the second part of the cycle. In this way, the patient's breathing can be artificially continued when needed by the patient. The one or more sensors 109 can monitor the cycles of the station 110 and capture information for the various parameters discussed above. This data can be communicated to the controller 101 for use in determining whether there needs to be any adjustment to the configurations of one or more components of the station 110.

As stated above, the second part of a breathe cycle comprises the scalable ventilator system 100 pulling the air out of the patient's lungs (i.e., performing the exhale action that the patient may not be able to perform). The negative air valve 104 is configured to couple to the patient through the manifold 105. When the air needs to be removed, the negative air valve 104 can open such that air from the patient's lungs can be pulled through the negative air valve 104. The air pulled from the patient's lungs through the input of the negative air valve 104 is output into the negative air supply 102b. Each cycle of the station 110 constitutes one breath of the patient.

In various embodiments, each station 110 can include a panel 113 that is communicatively coupled to the controller 113. The panel 113 serves as a local control panel for operation of the components of the station 110, where a medical professional is capable of making adjustments to the patient's treatment plan and/or the configuration of one or more components of the station 110. In some embodiments, a patient treatment plan can be entered into the panel 113 and saved to a non-transitory machine readable memory (not shown in FIG. 1) associated with the scalable ventilator system 100 and configured to maintain a record of one or more patient treatment plans. In some embodiments, the panel 113 can be used to make changes to a previously saved treatment plan, and the changes can be saved in the specific memory. In some embodiments, the panel 113 can include a local non-transitory machine-readable memory for storing a record of patient treatment plans associated with the patient. In this way, a medical professional can review previous characteristics of the treatment plan for use in evaluating progress and/or diagnosis. In some embodiments, the panel 113 may be capable of maintaining treatment plans for more than one patient, each plan being tagged with metadata identifying the specific patient. This can allow the medical professional to maintain records of more than one patient on the panel 113 so that the usage history can be maintained as patients connected to the station change. In other embodiments, the panel 113 may be configured to view old treatment plans for the current patient and/or treatment plans for other patients by requesting the plan from the memory of the scalable ventilator system 100 through the controller 101.

As stated above, the technology disclosed herein provides an easily scalable system for ventilation compared to commercial ventilators. The controller 101 is capable of controlling the operation of a plurality of stations. In this way, the same hardware and software circuitry can be used to control a plurality of different stations, as opposed to current ventilation systems that are each contained medical units. This reduces the scalability of commercial ventilators, making it difficult to ensure enough supply in view of large scale pandemics. Moreover, the controller 101 is capable of scaling based on the specific need, each controller 101 configured to control the operation of a plurality of ventilators, dependent on the availability of inputs and outputs that can be communicatively coupled to the controller 101. In various embodiments, each scalable ventilator system, like the system 100 discussed with respect to FIG. 1, can operate upwards of a 64 stations simultaneously, each station being individually controllable. Independent control allows for each station to be operated differently compared to other stations, unlike current dual-capacity ventilators that require both patients being treated are on the same treatment scheme.

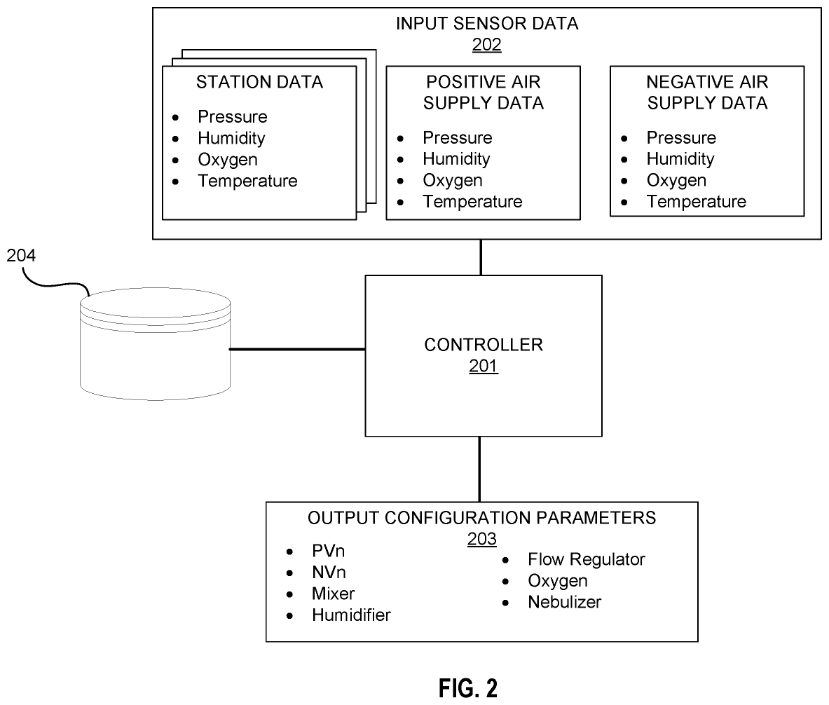

FIG. 2 shows an example controller flow diagram 200 in accordance with embodiment of the technology disclosed herein. The controller flow diagram 200 is provided for illustrative purposes only and should not be interpreted as limiting the scope of the present disclosure. Where the same or similar references are common between figures, all discussion of the reference with association to any figure is equally applicable to all other instances of the reference unless otherwise stated. As shown in FIG. 2, the controller 201 is configured to receive a plurality of inputs 202. The controller 201 can be similar to the controller 101 discussed above with respect to FIG. 1. Referring back to FIG. 2, the controller 201 can be configured to receive a plurality of operational information from a plurality of sensors disposed within the scalable ventilator system, from each station connected to the controller 201. The types of sensor data received can include, but are not limited to, the air pressure within the station, the temperature within the station, the humidity of the air mixture in the station, and the oxygen level in the air mixture of the station, among others. Nothing should be interpreted as limiting the scope of the technology to only the identified sensor data categories. In addition to receiving this information associated with each station, the controller 201 is further configured to receive the same or similar data from a plurality of sensors configured to monitor the positive air supply and the negative air supply, respectively. The input sensor data 202 can be collected periodically, collected at the end of each breath cycle, through a request received from the controller 201, or when the one or more sensors detect a change in one or more parameters, among others.

The controller 201 can be configured to utilize the input sensor data 202 to determine the operational parameters at each station and determine if any changes need to be made to the configuration of the component of the respective station in response to the patient's treatment requirements. The controller 201 can be configured in various embodiments to execute one or more determination process instructions stored on a non-transitory machine-readable memory of the controller 201 (not shown in FIG. 2). In various embodiments, the one or more determination process instructions can take the input sensor data 202 of the specific station and the input sensor data 202 from each of the positive air supply and negative air supply, respectively. By accounting for the current state of the positive air supply and the negative air supply, the controller 201 can determine what adjustments need to be made based on the current state of the system overall. The controller 201 can compute a current operational state of the respective station and compare it against a patient treatment record stored in a treatment database 204. The treatment database 204 can comprise a non-transitory machine-readable storage media configured to store a plurality of treatment plans for the plurality of patients served by the scalable ventilator system associated with the controller 201. In some embodiments, the comparison of the computed operational state of the station and the treatment plan of the treatment database 204 associated with the patient serviced by the station can be performed by comparing the computed station operation against an entry in a lookup table of the treatment database 204. In various embodiments, the controller 201 can compute one or more of an air pressure, breathing volume, breathing cycle, time of a breath cycle, tidal volume, among others.

In response to determining that the computed station operational status does not meet the requirements of the patient's treatment, the controller 201 can determine one or more changes that need to be made to the operation of one or more components of the station. The need for one or more changes can be determined where the computed values indicative of the operational state fall outside of a specific threshold range. Each threshold range may be determined based on the impact of a given operational parameter on the functioning of the station with respect to the stored treatment plan. The controller 201 can include non-transitory machine-readable instructions that, when executed, cause the controller 201 to determine which components need to be reconfigured based on the results of the comparison. The determination can utilize one or more of the input sensor data received by the controller 201 such that the determination accounts for the overall state of the system. This enables the determination for each station to account for changes made since the last time the station's configuration changed. In various embodiments, the controller 201 can loop through all of the connected stations in making the determination, analyzing one station at a time in succession. In other embodiments, the controller 201 can be configured to analyze one or more stations in the system at the same time.

Where a change is to be made, the controller 201 is configured to create output configuration parameters 203. The output configuration parameters 203 can include one or more instructions to one or more components for reconfiguration of the respective component. As a non-limiting example, the controller 201 could determine that the humidity within the air mixture is too low, and the controller 201 can send an instruction to a humidifier (not shown in FIG. 2) to inject additional humidity into the air mixture. Some non-limiting examples of components for which the output configuration parameters 203 can be determined include the positive gas valve, the negative gas valve, the mixer, the humidifier, the flow regulator, the oxygen supply, the nebulizer, among others.

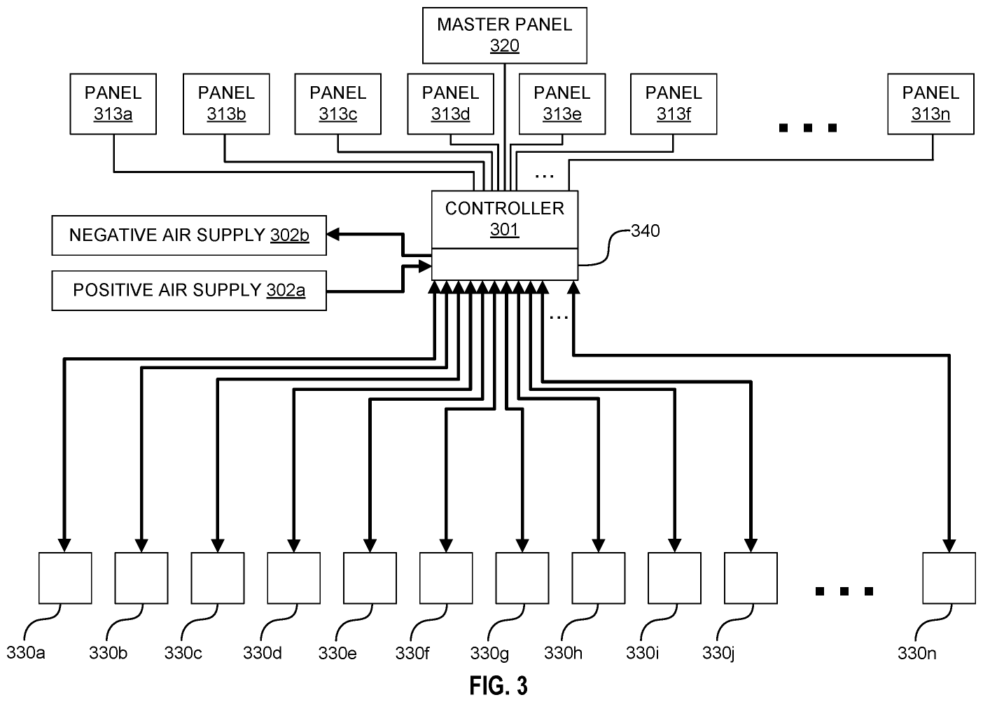

As discussed above, the technology disclosed herein provides a ventilation system that is scalable such that a plurality of different stations can be controlled by the same controller. FIG. 3 illustrates an example scalable ventilator system 300 comprising a plurality of stations 110 in accordance with embodiments of the technology disclosed herein. The example scalable ventilator system 300 is provided for illustrative purposes only. The scalable ventilator system 300 is similar to the scalable ventilator system 100 discussed with respect to FIG. 1, and should be interpreted as including the same or similar components as those discussed with respect to FIG. 1 unless otherwise stated. The system 300 includes a controller 301. The controller 301 can be similar to the controllers 101 and 201 discussed with respect to FIGS. 1 and 2. In various embodiments, the controller 301 can be configured to control the operation of a plurality of stations 330a-n simultaneously. Each station 330a-n can include the same or similar components as those of the station 110 discussed above with FIG. 100 above. Each station 330a-n can be individually controlled by the controller 301. In various embodiments, each of the stations 330a-n can be coupled to a master air supply manifold 340. The master air supply manifold 340 can comprise one or more manifold cavities, one for the positive air supply 302a and one for the negative air supply 302b. The positive air supply 302a and the negative air supply 302b can be similar to the air supplies 102a, 102b, respectively, discussed above with respect to FIG. 1. In various embodiments, the positive air supply 302a can comprise a plurality of positive air supplies, like positive air supply 102a, with each positive air supply 302a connected to a one or more of the positive air valves (not shown in FIG. 3) of one or more respective stations 330a-n. Similarly, the negative air supply 302b can comprise a plurality of negative air supplies, like negative air supply 102b, with each negative air supply 302b connected to a one or more of the negative air valves (not shown in FIG. 3) of one or more respective stations 330a-n.

As discussed above with respect to FIGS. 1 and 2, the controller 301 can be communicatively coupled to the components of each station 330a-n, although the communication lines between the controller and the components of each station 330a-n are omitted to avoid confusion in FIG. 3. In various embodiments, the controller can be communicatively coupled to a plurality of panels 313a-n, each panel being associated with a respective station of stations 330a-n, similar to the panel 113 discussed with respect to FIG. 1. In some embodiments, the controller 301 can be communicatively coupled to one or more master panels 320. A master panel 320 is similar to the panels 113 discussed with respect to FIG. 1, but is not associated with a specific station. Rather, a master panel 320 is capable of controlling any of the stations 330a-n. In various embodiments, one or more master panels 320 can be disposed away from any specific station 330a-n. As a non-limiting example, a master panel 320 can be disposed at a nursing station on a floor of the hospital, with the master panel 320 configured to monitor all of the stations 330a-n connected to the respective controller 301, and to enable any of the stations 330a-n to be controlled and manipulated by a medical professional from the nursing station instead of requiring local manipulation through the associated panel 313a-n of the specific station 330a-n. In various embodiments, one or more of the master panels 320 and/or the panels 313a-n can communicate with the controller 301 over a wired connection (as a non-limiting example, Ethernet), while in other embodiments the communication can be facilitated over a wireless connection (as a non-limiting example, WiFi, Bluetooth).

As discussed above, the scalable ventilator system can scale to accommodate a plurality of stations, thereby increasing the number of patients that can be serviced and controlled from the same controller. Moreover, rather than requiring an air supply for each station or each two stations, the scalable ventilator system enable multiple stations to be serviced by the same positive and negative air supply. Accordingly, the technology disclosed herein provides greater flexibility in the face of a pandemic or other disaster when the supply of commercially available ventilators cannot keep up with the need. Moreover, in some embodiments, the scalable ventilator system can be built into the hospital (either originally or through retrofitting) such that a plurality of stations can be available for use in emergency situations without the need to bring the controller into the hospital room as an external device. As a non-limiting example, a centralized scalable ventilator controller rack can be disposed on each floor, with one or more controllers installed therein. Each controller can be configured to service a plurality of stations throughout the floor. In this way, in the event of an emergency, the stations can be utilized without the need to set up the scalable ventilator system.

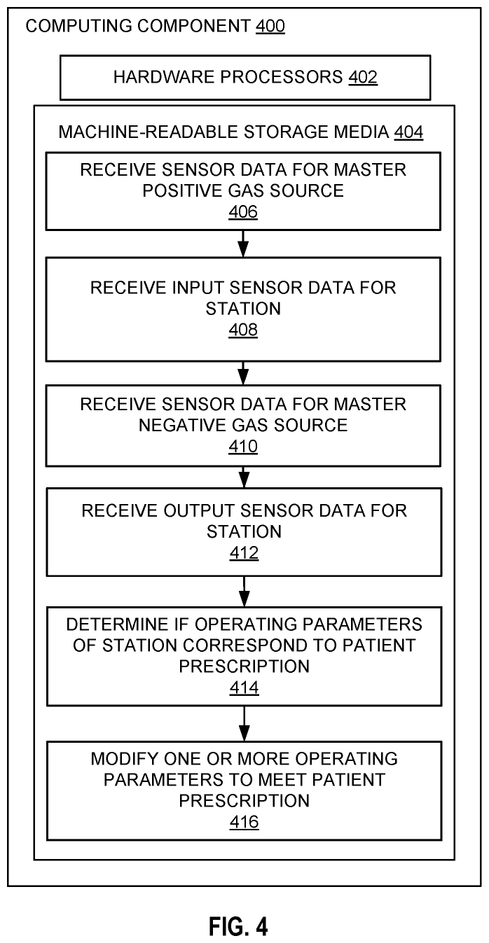

FIG. 4 is an example computing device 400 in accordance with embodiments of the present disclosure. Where operations and functionality of computing device 400 are similar to those discussed with respect to FIGS. 1-3, the description should be interpreted to apply. In various embodiments, the computing device 400 may be the controller 101, 201, or 301 discussed with respect to FIGS. 1-3. The computing device 400 includes hardware processors 402. In various embodiments, hardware processors 402 may include one or more processors.

Hardware processors 402 are configured to execute instructions stored on a machine-readable medium 404. Machine readable medium 404 may be one or more types of non-transitory computer storage mediums. Non-limiting examples include: flash memory, solid state storage devices (SSDs); a storage area network (SAN); removable memory (e.g., memory stick, CD, SD cards, etc.); or internal computer RAM or ROM; among other types of computer storage mediums. In various embodiments, the machine readable medium 404 can be similar to the memory and/or database 204 discussed with respect to FIG. 2. The instructions stored on the machine-readable medium 404 may include various sub-instructions for performing the function embodied by the identified functions. For example, the instructions “receive sensor data for master positive gas source” 406 may include various sub-instructions for receiving sensor data associated with the positive gas supply in a manner similar to that discussed above with respect to FIGS. 1-3. The instruction “receive input sensor data for station” 408 may include various sub-instructions for receiving sensor data associated with the input components of a specific station of the plurality of stations in a manner similar to that discussed above with respect to FIGS. 1-3. The instruction “receive sensor data for master negative gas source” 410 may include various sub-instructions for receiving sensor data associated with the negative master gas supply in a manner similar to that discussed above with respect to FIGS. 1-3. The instruction “receive output sensor data for station” 412 may include various sub-instructions for receiving sensor data associated with the output components of the specific station in a manner similar to that discussed above with respect to FIGS. 1-3.

The instruction “determine if operating parameters of station correspond to patient treatment plan” 414 may include sub-instructions for computing one or more parameters concerning the operational status of the specific station, the positive air supply, and the negative air supply. The instruction 414 may further include sub-instructions for comparing the computed parameters against a treatment plan associated with the patient be treated at the station to determine if the computed parameters are consistent with the patient's treatment plan. The instruction 414 may further include sub-instructions to generate one or more configuration changes for one or more components of the scalable ventilator system to bring the operational status of the station into compliance with the patient's treatment plan. The instruction “modify one or more operating parameters to meet patient prescription” 416 can include sub-instructions for communicating the change in configuration for the one or more components based on the generated configuration changes. The instruction 416 can further include sub-instructions for determining over what channel to communicate the changed configuration parameters.

It should be noted that the terms “optimize,” “optimal” and the like as used herein can be used to mean making or achieving performance as effective or perfect as possible. However, as one of ordinary skill in the art reading this document will recognize, perfection cannot always be achieved. Accordingly, these terms can also encompass making or achieving performance as good or effective as possible or practical under the given circumstances, or making or achieving performance better than that which can be achieved with other settings or parameters.

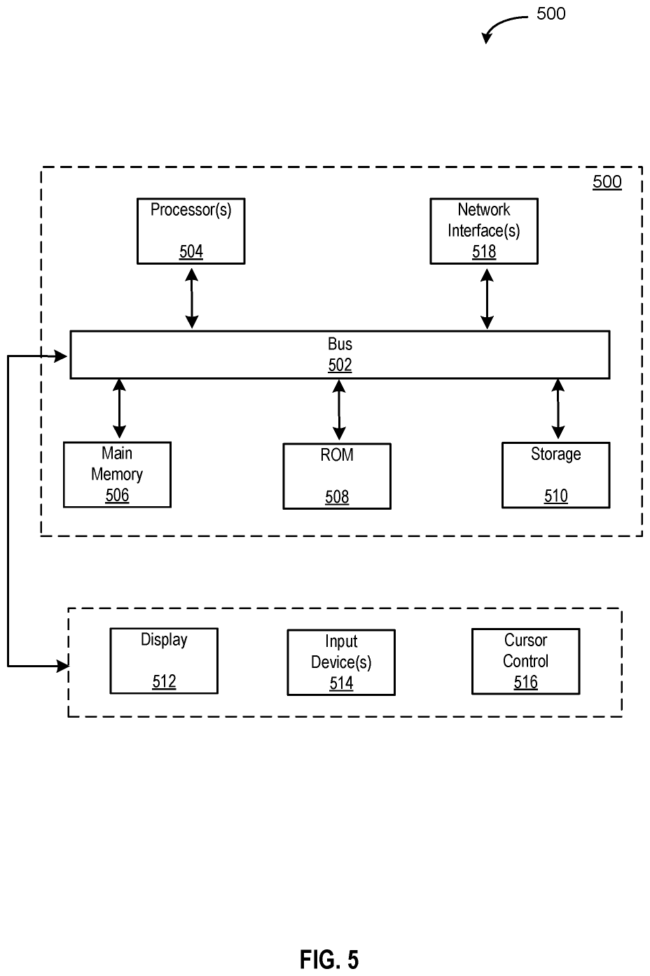

FIG. 5 depicts a block diagram of an example computer system 500 in which various of the embodiments described herein may be implemented. The computer system 500 includes a bus 502 or other communication mechanism for communicating information, one or more hardware processors 504 coupled with bus 502 for processing information. Hardware processor(s) 504 may be, for example, one or more general purpose microprocessors.

The computer system 500 also includes a main memory 506, such as a random access memory (RAM), cache and/or other dynamic storage devices, coupled to bus 502 for storing information and instructions to be executed by processor 504. Main memory 506 also may be used for storing temporary variables or other intermediate information during execution of instructions to be executed by processor 504. Such instructions, when stored in storage media accessible to processor 504, render computer system 500 into a special-purpose machine that is customized to perform the operations specified in the instructions.

The computer system 500 further includes a read only memory (ROM) 508 or other static storage device coupled to bus 502 for storing static information and instructions for processor 504. A storage device 510, such as a magnetic disk, optical disk, or USB thumb drive (Flash drive), etc., is provided and coupled to bus 502 for storing information and instructions.

The computer system 500 may be coupled via bus 502 to a display 512, such as a liquid crystal display (LCD) (or touch screen), for displaying information to a computer user. An input device 514, including alphanumeric and other keys, is coupled to bus 502 for communicating information and command selections to processor 504. Another type of user input device is cursor control 516, such as a mouse, a trackball, or cursor direction keys for communicating direction information and command selections to processor 504 and for controlling cursor movement on display 512. In some embodiments, the same direction information and command selections as cursor control may be implemented via receiving touches on a touch screen without a cursor.

The computing system 500 may include a user interface module to implement a GUI that may be stored in a mass storage device as executable software codes that are executed by the computing device(s). This and other modules may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

In general, the word “component,” “engine,” “system,” “database,” data store,” and the like, as used herein, can refer to logic embodied in hardware or firmware, or to a collection of software instructions, possibly having entry and exit points, written in a programming language, such as, for example, Java, C or C++. A software component may be compiled and linked into an executable program, installed in a dynamic link library, or may be written in an interpreted programming language such as, for example, BASIC, Perl, or Python. It will be appreciated that software components may be callable from other components or from themselves, and/or may be invoked in response to detected events or interrupts. Software components configured for execution on computing devices may be provided on a computer readable medium, such as a compact disc, digital video disc, flash drive, magnetic disc, or any other tangible medium, or as a digital download (and may be originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware components may be comprised of connected logic units, such as gates and flip-flops, and/or may be comprised of programmable units, such as programmable gate arrays or processors.

The computer system 500 may implement the techniques described herein using customized hard-wired logic, one or more ASICs or FPGAs, firmware and/or program logic which in combination with the computer system causes or programs computer system 500 to be a special-purpose machine. According to one embodiment, the techniques herein are performed by computer system 500 in response to processor(s) 504 executing one or more sequences of one or more instructions contained in main memory 506. Such instructions may be read into main memory 506 from another storage medium, such as storage device 510. Execution of the sequences of instructions contained in main memory 506 causes processor(s) 504 to perform the process steps described herein. In alternative embodiments, hard-wired circuitry may be used in place of or in combination with software instructions.

The term “non-transitory media,” and similar terms, as used herein refers to any media that store data and/or instructions that cause a machine to operate in a specific fashion. Such non-transitory media may comprise non-volatile media and/or volatile media. Non-volatile media includes, for example, optical or magnetic disks, such as storage device 510. Volatile media includes dynamic memory, such as main memory 506. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, and networked versions of the same.

Non-transitory media is distinct from but may be used in conjunction with transmission media. Transmission media participates in transferring information between non-transitory media. For example, transmission media includes coaxial cables, copper wire and fiber optics, including the wires that comprise bus 502. Transmission media can also take the form of acoustic or light waves, such as those generated during radio-wave and infra-red data communications.

The computer system 500 also includes a communication interface 518 coupled to bus 502. Network interface 518 provides a two-way data communication coupling to one or more network links that are connected to one or more local networks. For example, communication interface 518 may be an integrated services digital network (ISDN) card, cable modem, satellite modem, or a modem to provide a data communication connection to a corresponding type of telephone line. As another example, network interface 518 may be a local area network (LAN) card to provide a data communication connection to a compatible LAN (or WAN component to communicated with a WAN). Wireless links may also be implemented. In any such implementation, network interface 518 sends and receives electrical, electromagnetic or optical signals that carry digital data streams representing various types of information.

A network link typically provides data communication through one or more networks to other data devices. For example, a network link may provide a connection through local network to a host computer or to data equipment operated by an Internet Service Provider (ISP). The ISP in turn provides data communication services through the world wide packet data communication network now commonly referred to as the “Internet.” Local network and Internet both use electrical, electromagnetic or optical signals that carry digital data streams. The signals through the various networks and the signals on network link and through communication interface 518, which carry the digital data to and from computer system 500, are example forms of transmission media.

The computer system 500 can send messages and receive data, including program code, through the network(s), network link and communication interface 518. In the Internet example, a server might transmit a requested code for an application program through the Internet, the ISP, the local network and the communication interface 518.

The received code may be executed by processor 504 as it is received, and/or stored in storage device 510, or other non-volatile storage for later execution.

Each of the processes, methods, and algorithms described in the preceding sections may be embodied in, and fully or partially automated by, code components executed by one or more computer systems or computer processors comprising computer hardware. The one or more computer systems or computer processors may also operate to support performance of the relevant operations in a “cloud computing” environment or as a “software as a service” (SaaS). The processes and algorithms may be implemented partially or wholly in application-specific circuitry. The various features and processes described above may be used independently of one another, or may be combined in various ways. Different combinations and sub-combinations are intended to fall within the scope of this disclosure, and certain method or process blocks may be omitted in some implementations. The methods and processes described herein are also not limited to any particular sequence, and the blocks or states relating thereto can be performed in other sequences that are appropriate, or may be performed in parallel, or in some other manner. Blocks or states may be added to or removed from the disclosed example embodiments. The performance of certain of the operations or processes may be distributed among computer systems or computers processors, not only residing within a single machine, but deployed across a number of machines.

As used herein, a circuit might be implemented utilizing any form of hardware, software, or a combination thereof. For example, one or more processors, controllers, ASICs, PLAs, PALs, CPLDs, FPGAs, logical components, software routines or other mechanisms might be implemented to make up a circuit. In implementation, the various circuits described herein might be implemented as discrete circuits or the functions and features described can be shared in part or in total among one or more circuits. Even though various features or elements of functionality may be individually described or claimed as separate circuits, these features and functionality can be shared among one or more common circuits, and such description shall not require or imply that separate circuits are required to implement such features or functionality. Where a circuit is implemented in whole or in part using software, such software can be implemented to operate with a computing or processing system capable of carrying out the functionality described with respect thereto, such as computer system 500.

As used herein, the term “or” may be construed in either an inclusive or exclusive sense. Moreover, the description of resources, operations, or structures in the singular shall not be read to exclude the plural. Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps.

Terms and phrases used in this document, and variations thereof, unless otherwise expressly stated, should be construed as open ended as opposed to limiting. Adjectives such as “conventional,” “traditional,” “normal,” “standard,” “known,” and terms of similar meaning should not be construed as limiting the item described to a given time period or to an item available as of a given time, but instead should be read to encompass conventional, traditional, normal, or standard technologies that may be available or known now or at any time in the future. The presence of broadening words and phrases such as “one or more,” “at least,” “but not limited to” or other like phrases in some instances shall not be read to mean that the narrower case is intended or required in instances where such broadening phrases may be absent.

What is claimed is:

1. A scalable ventilator system, comprising:
multiple ventilator stations each comprising:
a positive air valve configured to receive air from a positive air supply,
a flow regulator configured to control a rate of flow of the air, and
a manifold configured to deliver the air to a patient; and
a controller configured to control the positive air valves and the flow regulators.

2. The scalable ventilator system of claim 1, wherein at least one of the ventilator stations further comprises:
one or more sensors configured to monitor the air, wherein the one or more sensors are communicatively coupled to the controller.

3. The scalable ventilator system of claim 1, wherein at least one of the ventilator stations further comprises:
a humidifier configured to control a humidity of the air, wherein the humidifier is controlled by the controller.

4. The scalable ventilator system of claim 1, wherein at least one of the ventilator stations further comprises:
a mixer configured to add one or more fluids to the air, wherein the mixer is controlled by the controller.

5. The scalable ventilator system of claim 1, wherein at least one of the ventilator stations further comprises:
a negative air valve configured to provide the air to a negative air supply, wherein the negative air valve is controlled by the controller.

6. The scalable ventilator system of claim 1, further comprising:
a master control panel configured to control the controller according to user inputs.

7. The scalable ventilator system of claim 1, wherein at least one of the ventilator stations further comprises:
a local control panel configured to control a controller configured to control the positive air valve and the flow regulator of the at least one of the ventilator stations according to user inputs.

8. The scalable ventilator system of claim 1, wherein the controller comprises:
multiple slots each connected to one of the multiple stations.

9. The scalable ventilator system of claim 8, wherein the controller further comprises:
multiple additional slots each configured to connect to a respective additional station.

10. A scalable ventilator system, comprising:
multiple ventilator stations each comprising:
a positive air valve,
a flow regulator, and
a manifold configured to deliver air to a patient;
a hardware processor; and
a non-transitory machine-readable storage medium encoded with instructions executable by the hardware processor to perform operations comprising:
operating the positive air valve to receive the air from a positive air supply, and
operating the flow regulator to control a rate of flow of the air to the manifold.

11. The scalable ventilator system of claim 10, wherein the operations further comprise:
receiving sensor data concerning the air from one or more sensors in at least one of the ventilator stations.

12. The scalable ventilator system of claim 10, wherein the operations further comprise:
operating a humidifier to control a humidity of the air in at least one of the ventilator stations.

13. The scalable ventilator system of claim 10, wherein the operations further comprise:
operating a mixer to add one or more fluids to the air in at least one of the ventilator stations.

14. The scalable ventilator system of claim 10, wherein the operations further comprise:
operating a negative air valve to provide the air to a negative air supply in at least one of the ventilator stations.

15. The scalable ventilator system of claim 10, wherein the operations further comprise:
operating the positive air valves and the flow regulators in the ventilator stations according to user inputs at a master control panel.

16. The scalable ventilator system of claim 10, wherein the operations further comprise:
operating the positive air valve and the flow regulator in one of the ventilator stations according to user inputs at a local control panel.

17. A computer-implemented method, comprising:
operating positive air valves in multiple ventilator stations to provide air from a positive air supply to each of the multiple ventilator stations;
receiving sensor data concerning the air from sensors in the multiple ventilator stations; and
operating flow regulators in the in the multiple ventilator stations to control rates of flow of the air to respective manifolds in the multiple ventilator stations in accordance with the sensor data and respective patient treatment plans.

18. The computer-implemented method of claim 17, further comprising at least one of:
operating a humidifier to control a humidity of the air in at least one of the ventilator stations; and
operating a mixer to add one or more fluids to the air in at least one of the ventilator stations.

19. The computer-implemented method of claim 17, further comprising:
operating the positive air valves and the flow regulators in the ventilator stations according to user inputs at a master control panel.

20. The computer-implemented method of claim 17, further comprising:
operating the positive air valve and the flow regulator in one of the ventilator stations according to user inputs at a local control panel.

21. A scalable ventilator system, comprising:
a plurality of ventilator modules;
a controller to control operation of the plurality of ventilator modules; and
a station module comprising a plurality of receptacles, wherein each receptacle is configured to accept one of the plurality of ventilator modules;
wherein each ventilator module comprises a plurality of solenoid valves and an input hose and an output hose, wherein each ventilator is controlled individually to provide individualized regimens based on needs of a patient corresponding to a respective ventilator.

22. The scalable ventilator system of claim 21, wherein a quantity of ventilator modules installed into their respective receptacles in the station module may be scaled to meet patient requirements.
